(19) United States
(12) Patent Application Publication
(10) Pub. No.: US 2015/0125866 A1
(43) Pub. Date: May 7, 2015

(54) METHOD FOR SELECTING DONORS AND RECIPIENTS FOR TRANSPLANTATION

(71) Applicant: NHS Blood & Transplant, Watford, Hertfordshire (GB)

(72) Inventor: John Harvey, Filton Bristol (GB)

(21) Appl. No.: 14/397,510

(22) PCT Filed: May 3, 2013
(86) PCT No.: PCT/GB2013/051160

§ 371 Date: Oct. 28, 2014

Related U.S. Application Data
(60) Provisional application No. 61/642,608, filed on May 4, 2012.

(30) Foreign Application Priority Data
May 4, 2012 (GB) 1207831.7

(51) Int. Cl. C12Q 1/68

(52) U.S. Cl. CPC C12Q 1/6881
USPC 435/6.12; 435/287.2

(57) ABSTRACT

The present invention discloses a method for determining a suitable donor for a recipient in need of transplant based on identifying short tandem repeat allele length differences.

The present invention relates to a method for selecting a donor for a recipient in need of a transplant with materials such as organs, cells or tissues from a donor. In particular, the invention relates to identifying donor and recipient pairs at higher risk of recipient mortality in allogeneic haematopoietic progenitor cell transplantations (HPCT) or graft loss in solid organ transplantation

Despite major advances in MHC immunogenetics through DNA-based Human Leucocyte Antigen (HLA) typing technologies and improved matching, allogeneic hematopoietic progenitor cell transplantation (HSCT) is still associated with a significant risk of acute graft versus host disease (aGVHD), chronic GVHD and mortality¹. Prof. John Hansen² states “Despite complete matching for all variation spanning 4 Mb of DNA across the MHC, acute GVHD and transplant related mortality (TRM) occurs in a significant number of HLA identical donor transplants.” Hansen goes on to review genetic polymorphism in other genes reported to be associated with progenitor cell transplant outcomes. He lists them as IL2A, IL1B, IL 1RN, IL6, IL10, INFG, TGFB, TNF, CTLA4, ESR1, IL2, IL7, IL8, IL 10RB, IL18, NOD2 and VDR. The results of these single centre studies have not been independently validated by others in separate patient populations and that the original results lack statistical power due to the small number of cases.

Other workers have similarly extensively reviewed many of the factors reviewed by Hansen but also included known minor histocompatibility factors such as HA-1, CD31 and NK cell reactivity and the KIR gene locus and ligands. Mulligan and Brady³ suggest that none of these non-HLA markers are ready for routine use as clinical tools without further studies to address their limitations. They further state that studies of these factors have failed to produce a clinically useful model for risk and the published studies have at times been conflicting. Mulligan and Brady also state that it is conceptually attractive to have a single genetic variant assist in clinical decision making in the HLA matched stem cell transplant setting, but that this is likely to be over simplistic. They state that what is required is a risk “index” of clinical and genetic variables that distinguishes between multiple potential donors for patients, highlighting potential transplant pairs at highest risk of complications.

Using Microsatellite (Msats or Short Tandem Repeat [STR]) markers in population genetic structure studies it has been found that genetic differences between individuals from varied human populations only slightly exceed those found between individuals from a single population. Within population genetic variation, accounts for ˜95% of human genetic diversity. Europe is noted to have the lowest among population molecular variance estimated at 0.7% (95% C.I.; 0.6-0.9) compared to 11.6% for America (95% C.I.; 11-12.3)⁴. It is likely that this risk index will have differing impacts in differing population groups with the Western European population having the lowest levels of variation. The challenge for genetic studies in HPCT is to identify genetic variations that impact transplant outcome from the relatively small amount of genetic differentiation that exists between individuals.

Unlike most other forms of genomic polymorphism, Msats loci have the characteristic of variable nucleotide repeat unit length. Differences in repeat unit length relates to ancestral divergence on a time related basis. Slatkin⁵ used the differences in Msats allele length found in differing populations to derive a measure for genetic distance termed R_{ST}.

Patient/donor Msat incompatibilities have also been used as surrogate markers to map biologically relevant polymorphisms, with a main focus on MHC genetic variation. Several studies have used Msat alleles as surrogate markers to assess patient/donor compatibility in unrelated HSCT. Li et al.⁶ have analysed 13 Msat loci in 100 HLA-A,B,C,DRB1,DQB1 allele-matched patient/donor pairs and did not find any significant association with clinical outcome. Using the data set of the International Histocompatibility Working Group (IHWG) in HSCT, Malkki et al.⁷ have reported that mismatching at five Msat markers (D6S105, D6S265 and D6S2811 in the class I region, D6S2787 in the class III region and D6S2749 in the class II region) was associated with GVHD incidence or mortality in a cohort of 819 HLA-A,B,C,DRB1,DQB1 allele-matched HSCT pairs. In a recent review of Msat's, Tiercy⁸ states that “among the trends starting to emerge from these studies, specific TNFd Msat alleles seem to be associated with acute graft-versus-host disease and mortality. Patient/donor Msat incompatibilities have also been used as surrogate markers to map biologically relevant polymorphisms, with a main focus on MHC-resident genetic variation.” Using the same 15 Msat's as used in this study Alcoceba et. al.⁹, showed that increased Msat mismatches in HLA matched sibling transplants resulted in increased aGVHD and mortality⁹. This data suggested that Msat could be informative to map non-HLA determinants of clinical importance in HSCT in the MHC.

Over 20 million voluntary unrelated stem cell donors have been HLA typed for donor registries worldwide. The largest single group being in the National Marrow Donor Panel, USA. Any genetic determinant identified between donors and recipients that significantly improve patient survival can be tested for and selected for prior to transplantation. At this time only HLA markers and blood groups are routinely matched for at the genetic level.

In haematopoietic progenitor cell transplantation one of the earliest steps in donor selection is to consider the disease and the potential progression of the patient. Patients with a slowly progressing disease such as myelodysplastic syndrome in low and intermediate-1 international prognostic score groups (IPSS), can have ample time to search for the best HLA matched related or unrelated donor. In these cases delaying transplantation to source the best possible donor can maximise overall survival. However, in others cases such as patients with myelodysplasia in intermediate-2 and high risk IPSS, immediate transplantation is associated with maximal life expectancy.

This contrasts with acute leukaemias where the patient's condition can rapidly deteriorate and a limited window of opportunity in terms of clinical remission may limit the time available for an unrelated donor search. The transplant physician must advise the HLA typing laboratory on the stage of the patient's disease (early, intermediate or advanced) giving an indication of clinical urgency. A patient progressing to advanced disease usually has a higher mortality risk from the disease than the added risk from a single HLA allele/antigen mismatch or alternative donor therapy such as umbilical cord blood (UCB) transplantation. The progress of the patient's disease and the likelihood of finding an HLA matched donor will determine the choice of progenitor cell source selected for treatment. However, even where a fully HLA matched donor is identified and is transplanted a significant number of patients still suffer from transplant related morbidity.

Previous studies have looked at polymorphism at single gene sites. None of these non-HLA (and non-ABO blood group) markers have resulted in clinical tools for routine use in the transplant setting.

According to the present invention there is provided a method for selecting a donor for a recipient in need of a transplant comprising the steps of taking a sample from the donor and recipient, extracting the DNA, measuring the short tandem repeat (STR) allele lengths in the donor and recipient DNA, determining the difference in length between the donor and recipient alleles and selecting the donor based on the difference in length.

Preferably, the donor and recipient are HLA matched.

The donor and recipient STR alleles may be paired according to length, prior to determining the difference in length.

Conveniently, the donor may be selected based on the smallest difference in STR allele lengths in a given donor/recipient STR allele pair.

According to another aspect of the invention there is provided a method of selecting a donor for a recipient in need of a transplant comprising the steps of measuring the short tandem repeat allele sizes of the donor and recipient, assigning numerical weightings to the allele size differences for each of the donor and recipient pairs, comparing the weightings to provide a value, determining whether the value is at, above or below a pre-determined threshold value and thereby selecting the donor having the appropriate value.

Weight average allelic size difference (WAASD) will hence forth be referred to as Average allelic length discrepancy (AALD) and is the same as or equivalent measure to WAASD.

The method could be performed in addition to HLA matching of donor and recipient as its effect is additional and independent from HLA match criteria. In one embodiment, the higher the Average Allelic Length Discrepancy (AALD) weighting, the greater the risk of increased mortality of the recipient in haematopoietic progenitor cell transplants or organ failure for solid organ transplants, following transplantation. Preferably, the weightings may be calculated using the following formula:—

$$AALD = \frac{\sum ( a 1 \cdot b 1 + a 2 \cdot b 2 + a 3 \cdot b 3 + … )}{\sum f}$$

a1=the first allelic size difference observed, b1=the frequency by which value a1 is observed
a2=the second allelic size difference observed, b2=the frequency by which value a2 is observed
a3=the third allelic size difference observed, b3=the frequency by which value a3 is observed etc. . . .

Σ(sum)f=(b1+b2+b3 . . . ) for frequency values observed

The number of a. values used in the formula is dependent upon the observed allelic size differences noted between the recipient and donor and will vary among transplant pairs. The b. values are determined by the observed frequency of a given a. value for the donor and recipient being assessed. The f value is the sum (Σ) of all the b. values.

The AALD values identified in the present invention are designed to identify which HLA matched and mismatched donor/recipient pairs are most at risk of added morbidity due to probabilistic increased genomic differences. The AALD offers a tool assisting the transplanter to identify which HLA matched and mismatched donors are most likely to offer a lower risk of post-transplant mortality.

Preferably the donor selected is the one from the pair with the lowest difference in STR length, i.e. the lower AALD.

According to another aspect of the invention there is provided a method of determining recipient increased relative risk for morbidity following a transplant comprising the steps of measuring the short tandem repeat allele sizes of the donor and recipient, assigning numerical weightings to the allele size differences for each of the donor and recipient pairs, comparing the weightings to provide a value, and determining the recipient morbidity based on whether the value is at, above or below a pre-determined threshold value.

According to a further aspect of the invention there is provided a method of selecting a donor for a transplant comprising the steps of measuring the short tandem repeat allele sizes of the donor with a recipient, assigning numerical weightings to the allele size differences for the donor and recipient pairs, comparing the weightings to provide a value, and determining the recipient relative risk of morbidity based on the value and selecting the recipient with the lower risk of morbidity

According to another aspect of the invention there is provided a method of matching a donor with a recipient for a transplant comprising the steps of measuring the short tandem repeat allele sizes of the donor and recipient, pairing the STR alleles according to size, determining the size difference in each pair, assigning numerical weightings to the allele size differences for the donor and recipient pairs, comparing the weightings to provide a value, determining whether the value is at or below a threshold value indicative of a probabilistic improved genomic match with the donor and the recipient.

Conveniently, the value generated is compared to a threshold value in order to provide an indication of the optimum predicted survivability of the recipient.

Preferably the threshold value may be 1.7 and above when the donor and recipient are unrelated and have been HLA matched. Particularly where there is a racially mixed population cohort, the threshold value may be significantly higher. More preferably, the threshold value may range from 1.0 to 3.0 or higher. The threshold value range may be between 1.7 and 2. The threshold value can be predetermined.

If the value determined is above a threshold value, then that is indicative of a lower survivability or higher morbidity of the recipient of an unrelated donor transplant.

If the value determined is at or below a threshold value, then that is indicative of a higher survivability or lower morbidity of the recipient of an unrelated donor transplant.

Preferably the determined value is at or below the threshold value which is indicative of a greater predicted survivability of the recipient of an unrelated donor transplant.

By unrelated it is meant not known to be a genetically related relative to the recipient and was identified through a register of willing stem cell donors.

By related it is meant to be a donor who is genetically related to the recipient such as a sibling, parent or first cousin.

Preferably the threshold value is in the range from 0.5 to 0.8 when the donor and recipient are related and have been HLA matched. If the value determined is below a pre-determined threshold value, then that is indicative of a lower survivability or higher morbidity of the recipient of a related donor transplant.

If the value determined is at or above a threshold value, then that is indicative of a higher survivability or lower morbidity of the recipient of a related donor transplant.

Preferably the determined value is above the threshold value which is indicative of a greater predicted survivability of the recipient of a related donor transplant.

The orientation of beneficial threshold values may differ in admixture populations where there exists a high degree of genetic mix from ancestral populations (i.e. African American and Hispanic populations).

The donor and recipient samples may be taken from saliva, blood, an organ, cells, tissues, umbilical cord blood cells and other haematopoietic progenitor cells from adult and paediatric donors.

The inventors have developed a genetic difference “risk index” based upon Msats allele size differences between donor and recipient. In one embodiment the higher score levels correlate with increased mortality among transplant recipients of unrelated donor stem cells.

The donor and recipient alleles are identified for each STR loci and the smallest sized allele of the donor is paired with the smallest allele for the recipient and the size difference expressed in STR motif repeat units is determined. The larger sized donor allele is paired with the larger recipient allele and the size difference is similarly determined in repeat units. Where for an STR locus there is homozygosity for either donor or recipient alleles (or both) then the homozygous allele size is taken to be both the smallest and the largest allele and the size difference in STR motif repeat units between donor and recipient alleles is similarly determined.

According to another aspect of the invention there is provided a device comprising a means for measuring the short tandem repeat allele length sizes of the donor and recipient, ordering the alleles in donor recipient pairs according to length, determining the difference in length between the pairs, assigning a value to the difference, determining threshold value ranges for the length differences, determining whether the length difference of a pair falls within the threshold value and thereby determining the appropriate donor for a recipient.

Preferably, the large sized donor alleles are paired with the large sized recipient alleles and the smallest sized donor alleles are paired with the smallest sized recipient alleles. The donor and recipient are preferentially matched when the determined value is below the threshold value. Preferably the device has a means for determining increased recipient morbidity or organ failure following transplantation with biological material from a donor based on the AALD value.

The donor material may be from blood, saliva, an organ, cells, tissues, umbilical cord blood cells and other haematopoietic progenitor cells from adult and paediatric donors.

Preferably the devise may be selected from a computer disc, compact disc, computer programme, computer software, digital video disc (DVD) or any other means suitable for carrying the means.

The means may be an algorithm, a mathematical formula, statistical information or any other equivalent means.

The means may be numerically weighted average allele length discrepancy for short tandem repeats from donors and transplant recipients.

The numerical weighting is selected as follows:—the selection of a donor is initially made on the basis of HLA compatibility, where more than one donor is available for selection, the AALD values can be used to score the donors and identify which donor has the least likelihood of increasing transplant related mortality (TRM). For unrelated HLA matched donors this would be the lowest AALD score. In HLA matched related donors, this scenario may be different as the choice of a higher score may enhance an anti-leukaemic effect and the AALD score would still remain below the scores observed for most unrelated HLA matched donors.

The inventors present for the first time a means for analysing overall survival in paediatric haematopoietic progenitor cell transplant (HPCT) patients placing them on a “risk index” continuum derived from AALD values. A total of 180 paediatric transplants were analysed where microsatellite (Msat) data was available for donor and recipient following post-transplant monitoring for patient chimerism. As the patient pairs AALD values increase on this risk index above a value of 1.8 we show that the mortality rate increases with the higher values and this is statistically significant.

The inventors have used the differences in the length of 15 highly informative Msats (high level of heterogeneity) to generate a score indicative of a probabilistic increase in genetic difference between donor and recipients of haematopoietic progenitor cell transplants. However the number of Msat loci used can be varied with the AALD principal still applied.

Individuals with shared ancestry were shown to have fewer differences in STR lengths than those with highly divergent ancestry. The AALD for STR alleles between stem cell donors and recipients were applied and used as a metric for genomic divergence between the donor and recipient.

Using the unique characteristics of Msats a novel “risk index” based upon the AALD of 15 Msats (in one embodiment) known to display high levels of heterozygosity has been developed and can be used to determine the relative risk of mortality of the recipient after a transplant. Relative risk is standard statistical measurement between groups at risk of an event, the event in this instance is death.

There are no published studies where average Msat allelic length differences have been used as a proxy for genomic difference between donors and recipients in allogeneic transplantation. In the retrospective haematopoietic progenitor cell transplant study the AALD score showed a strong correlation with transplant mortality. Individuals with shared ancestry were shown to have fewer differences in Msat lengths than those with highly divergent ancestry. The AALD for Msat alleles between stem cell donors and recipients were applied as a metric for genomic divergence between the donor and recipient ancestries. It can be assumed that over thousands of generations there will be a continuous growth in the genetic mutations observed between divergent populations. With Msat mutations this increase would be measurable as increasing differences in the length of the Msat alleles observed between individuals within the population groups. Homoplasty in the mutation process leads to a degree of inaccuracy in the model but the work of Slatkin⁵ indicates that when average values are used, the Msat. size difference remains an accurate tool to measure genetic distance. Mutation rates are significantly higher in Msat polymorphism, than those observed for other parts of the genome. Ramakrishnan & Mountain¹⁰ describe an average mutation rate of 0.0005 (1/2000 generations) for 377 human Msat. loci with an average number of Msat. alleles present per constellation of 10.8 having a standard deviation of 3.6.

The AALD between donor and recipient for 15 STR loci was examined and generated an index of values which was used as a proxy for genome wide genetic difference between donor and recipient. These values form a continuum of genetic variation for the transplant pairs examined. The higher values on this continuum associate with higher transplant mortality. The value at which the increased mortality reaches statistical significance is 1.8 in the patient cohort tested.

The method described in the present invention can be used by donor registries for testing donor STR profiles which information can then be used by transplant centres to estimate the genetic distance between donor and recipient ancestries (using the AALD values described in this document). It would be of commercial value to an enterprise to have their STR profiling products accepted and used for volunteer donor registry panel and patient profiling. These profiles are currently used downstream for post-transplant monitoring of chimerism. It is a valid proposition that a commercial company would want to lead in developing this new “upstream” application as it may result in further benefits accruing with their products also being preferentially used for post-transplant monitoring “downstream” in the HPCT process (giving a competitive advantage).

It is possible that as with complex diseases, multiple genes interact and effect outcomes. In the AALD score for genetic divergence the inventors have provided a tool that indicates a probabilistic approach to whether a donor and recipient are likely to show multiple polymorphic differences. The postulation for the use of AALD values is that the greater the level of genetic divergence between donor and recipient the higher the probability of post-transplant mortality.

Thus the present invention can also be used for estimating the relative genetic divergence between donor and recipient ancestries. The invention can also be used to reduce the risk of post-transplant complications influencing recipient mortality.

The invention will now be described in the following examples and accompanying drawings by way of illustration only in which:—

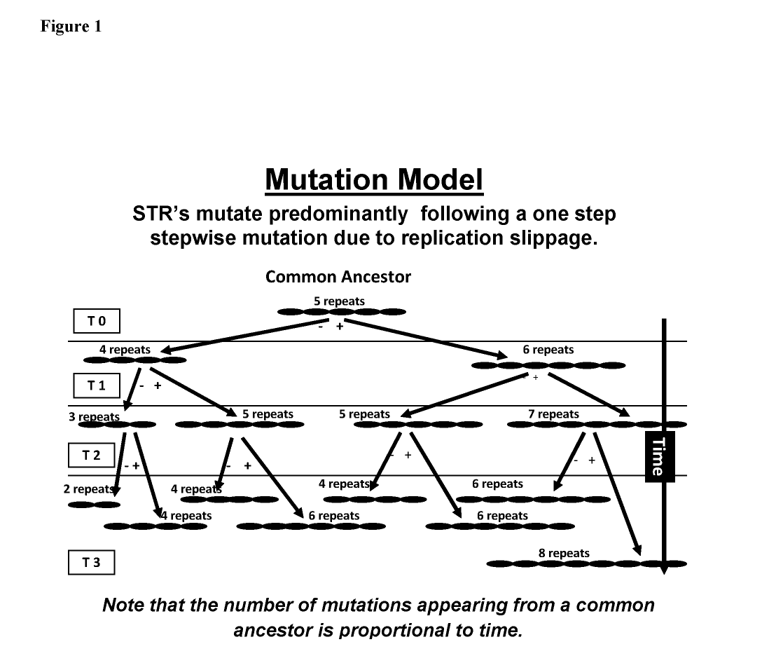

FIG. 1 is a flow diagram showing a step wise genetic mutation model for Microsatellites (Msat).

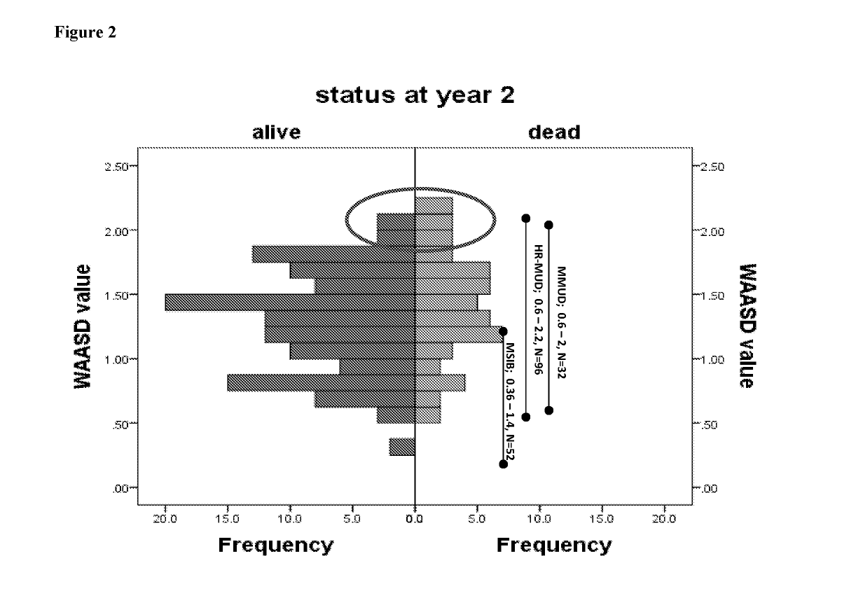

FIG. 2 is a graph showing the frequency of 180 paediatric alive and dead patients at differing points on the AALD (or WAASD) continuum. The term WAASD is used interchangeably with the term AALD.

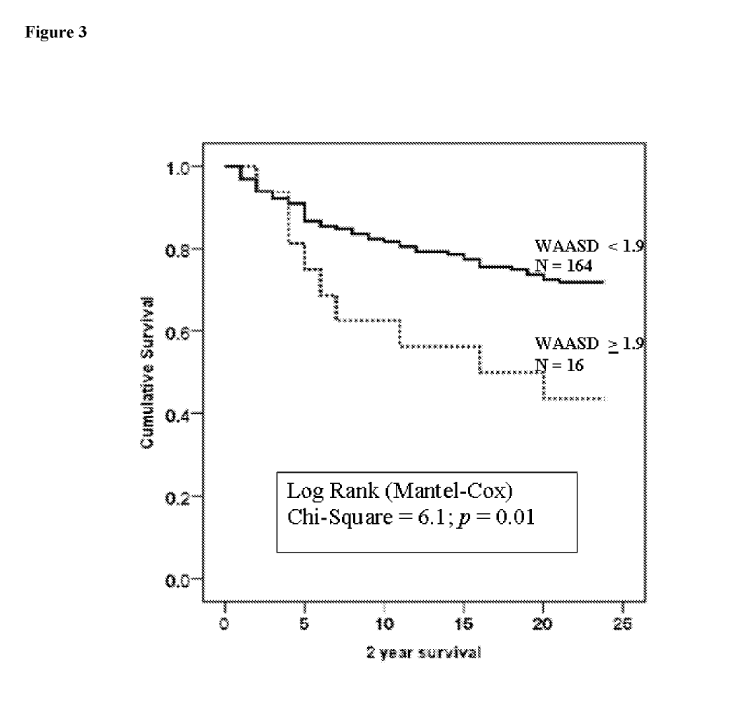

FIG. 3 shows a graph illustrating the Kaplan-Meier overall survival estimate for 180 paediatric transplant pairs with AALD (or WAASD) values above and below a value of 1.9. The term WAASD is used interchangeably with the term AALD.

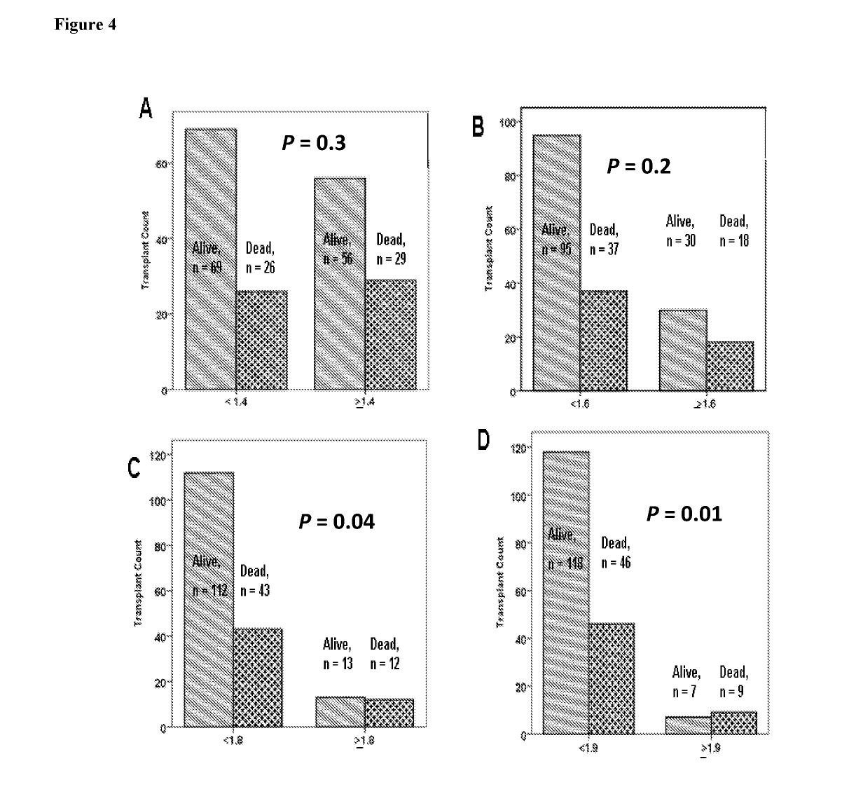

FIG. 4 (A to D) are histograms of death or survival at dichotomised AALD values from 180 consecutive paediatric transplants from a single transplant centre, transplanted for multiple disease causes.

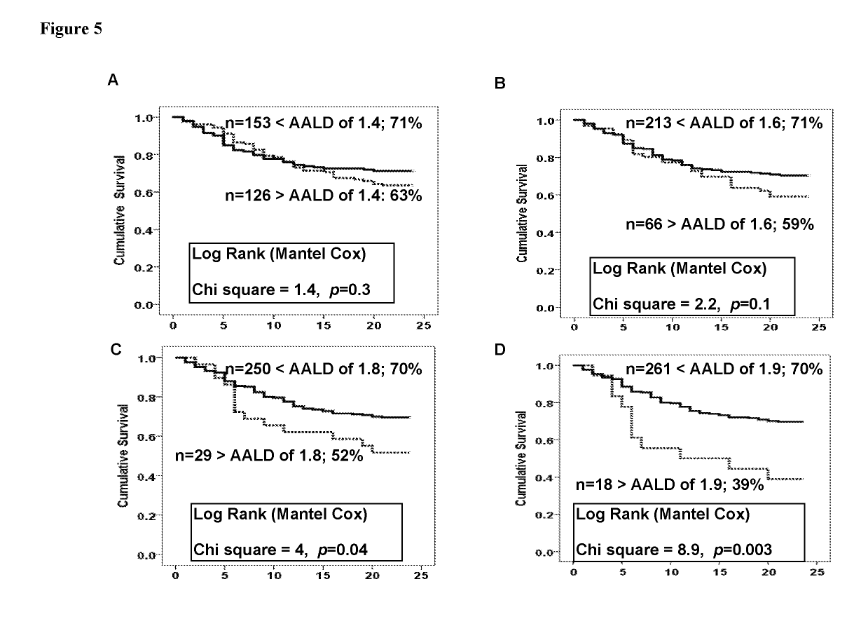

FIG. 5 (A to D) shows a graph illustrating the Kaplan-Meier overall survival estimate for 279 paediatric and adult transplant pairs with AALD values above and below four threshold values of 1.4, 1.6, 1.8 and 1.9. Patient 2 year survival of dichotomised values above and below AALD threshold scores of 1.4, 1.6, 1.8 and 1.9 for 279 Allogeneic Transplants.

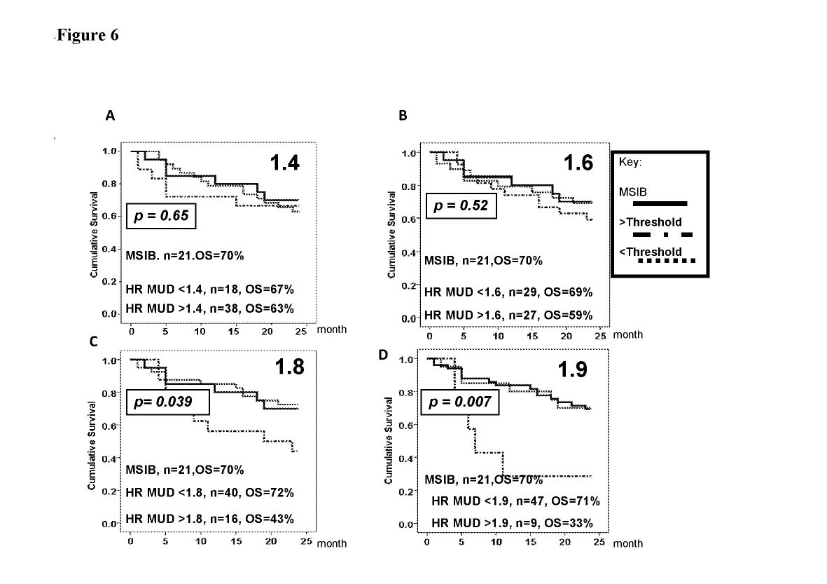

FIG. 6 (A-D) shows a graph illustrating the Kaplan-Meier overall survival estimate for a homogeneous cohort of 77 acute lymphoblastic leukaemic paediatric transplant pairs with AALD values above and below four threshold values of 1.4, 1.6, 1.8 and 1.9. The patients were HLA matched sibling (MSIB) outcomes compared with dichotomised AALD values at four thresholds—1.4; 1.6; 1.8; 1.9 for a homogeneously transplanted HR-HLA Matched Unrelated Donor paediatric ALL patient cohort

### EXAMPLES

### Example 1

### Patients

Polymorphisms of Msats are used routinely for post-transplant chimerism analyses in clinical HPCT (hematopoietic progenitor cell transplant). FIG. 1 shows a flow diagram showing a step wise genetic mutation model for Msat. In this diagram each oval shape represents a single Msat nucleotide repeat sequence AND depicts the effect of stepwise mutation over time. At mutation TO, there is only one ancestral allele consisting of five repeat DNA motifs. At each mutation step a repeat unit may be added or removed such that at mutation step T3 the possible number of alleles present in the population ranges from 2 repeat units to 8 repeat units. Six additional alleles have been introduced by step wise mutation in this model population.

The inventors have taken 180 paediatric HPCT's for which chimerism analysis is available and tabulated the donor and recipient Msats allele size. Multivariate analysis to test covariate influence on two year Overall Survival (OS) using logistic regression and the Cox proportional hazards model^{11;12} were performed using IBM, SPSS software (version 19). The following covariates were tested for possible confounding influence; disease status, patient/donor cytomegalovirus serology combinations, patient age, patient/donor relationship and HLA match grade, stem cell source, days to engraftment, patient/donor sex combinations, graft versus host disease and changes to pre-transplant conditioning protocol. This data is not reported here, but the AALD effect on overall patient survival was found to be independent of all listed covariates.

### Example 2

### AALD Determination

From the recipient and donor Msat profiles the inventors tabulated the Msat allele sizes for both recipient and donor for 15 Msat loci (see Table 1). The alleles were paired such that the smallest recipient allele was paired with the smallest donor allele and similarly with the larger alleles to obtain the recipient/donor allelic size difference (Table 1 & 2). Where the sizes were identical the size difference was assigned a value of zero. The inventors used the observed allele size difference in nucleotides to derive the number of Msat repeat nucleotide sequences by which the donor and recipient differ at each paired allele. Syngeneic twin AALD values were zero with all equivalent Msat alleles for donors and recipients being of the same length.

TABLE 1
An example of allele sizes observed in an HLA matched sibling
transplant pair for fifteen STR loci. A high level of shared
allele sizes were observed in sibling transplants.

| | Allele size | Allele size | Base pair | STR repeat unit |
|---|---|---|---|---|
| STR loci | Recipient | Donor | difference | motif difference |
| D3S1358 | 122 | 122 | 0 | 0 |
| | 126 | 126 | 0 | 0 |
| TH01 | 174 | 174 | 0 | 0 |
| | 174 | 174 | 0 | 0 |
| D21S11 | 216 | 232 | 16 | 4 |
| | 222 | 222 | 0 | 0 |
| D18S51 | 307 | 307 | 0 | 0 |
| | 311 | 303 | 8 | 2 |
| Penta E | 410 | 410 | 0 | 0 |
| | 440 | 410 | 30 | 6 |
| D5S818 | 129 | 129 | 0 | 0 |
| | 133 | 133 | 0 | 0 |
| D13S317 | 183 | 183 | 0 | 0 |
| | 195 | 195 | 0 | 0 |
| D7S820 | 220 | 220 | 0 | 0 |
| | 236 | 236 | 0 | 0 |
| D16S539 | 285 | 293 | 8 | 2 |
| | 293 | 293 | 0 | 0 |
| CSF1PO | 341 | 341 | 0 | 0 |
| | 353 | 353 | 0 | 0 |
| Penta D | 414 | 414 | 0 | 0 |
| | 420 | 400 | 20 | 4 |
| vWA | 146 | 146 | 0 | 0 |
| | 154 | 150 | 4 | 1 |
| D8S1179 | 213 | 213 | 0 | 0 |
| | 225 | 221 | 4 | 1 |
| TPOX | 269 | 269 | 0 | 0 |
| | 269 | 281 | 12 | 3 |
| FGA | 344 | 344 | 0 | 0 |
| | 348 | 348 | 0 | 0 |

TABLE 2
An example of allele sizes observed in an HLA matched unrelated
transplant pair for fifteen STR loci. A low level of shared
allele sizes was observed in unrelated transplants when
compared to sibling transplants (see Table 1).

| | Allele size | Allele size | Base pair | STR repeat unit |
|---|---|---|---|---|
| STR loci | Recipient | Donor | difference | motif difference |
| D3S1358 | 117 | 117 | 0 | 0 |
| | 126 | 122 | 4 | 1 |
| TH01 | 174 | 162 | 12 | 3 |
| | 174 | 166 | 8 | 2 |
| D21S11 | 222 | 214 | 8 | 2 |
| | 226 | 218 | 8 | 2 |
| D18S51 | 311 | 299 | 12 | 3 |
| | 310 | 322 | 12 | 3 |
| Penta E | 385 | 415 | 30 | 6 |
| | 400 | 420 | 20 | 4 |
| D5S818 | 129 | 129 | 0 | 0 |
| | 137 | 133 | 4 | 1 |
| D13S317 | 195 | 187 | 8 | 2 |
| | 199 | 191 | 8 | 2 |
| D7S820 | 219 | 219 | 0 | 0 |
| | 227 | 224 | 3 | 1 |
| D16S539 | 277 | 289 | 12 | 3 |
| | 293 | 289 | 4 | 1 |
| CSF1PO | 337 | 333 | 4 | 1 |
| | 337 | 333 | 4 | 1 |
| Penta D | 414 | 406 | 8 | 2 |
| | 414 | 406 | 8 | 2 |
| vWA | 138 | 146 | 8 | 2 |
| | 146 | 150 | 4 | 1 |
| D8S1179 | 229 | 225 | 4 | 1 |
| | 229 | 225 | 4 | 1 |
| TPOX | 269 | 273 | 4 | 1 |
| | 269 | 285 | 16 | 4 |
| FGA | 344 | 328 | 16 | 4 |
| | 352 | 356 | 4 | 1 |

The allelic size difference and the frequency of size difference as observed between the donor and recipient alleles were tabulated (Table 3.). The AALD between the donor and recipient was calculated from the sum product of the size difference multiplied by the frequency of the size difference observed, divided by the sum of the frequencies and tabulated.

TABLE 3
An example of the AALD for (A) an HLA matched sibling
pair and (B) an HLA matched unrelated transplant.
(A) Derived from table 1 HLA matched sibling transplant

| Repeat unit | | |
|---|---|---|
| differences observed | Frequency of observed | Average allele length |
| in table 1 | differences for table 1 | discrepancy for 15 STR |
| (a) | (b) | loci listed in table 1 |
| 0 | 22 | The sum product of column |
| | | (a) and (b) divided by the |
| | | sum of column (b). |
| | | AALD = 0.76 |
| 1 | 2 | |
| 2 | 2 | |
| 3 | 1 | |
| 4 | 2 | |
| 6 | 1 | |

(B) Derived from table 2 HLA matched unrelated transplant

| Repeat unit | | |
|---|---|---|
| differences observed | Frequency of observed | Average allele length |
| in table 2 | differences for table 2 | discrepancy for 15 STR |
| (a) | (b) | loci listed in table 2 |
| 0 | 3 | The sum product of column |
| | | (a) and (b) divided by the |
| | | sum of column (b). |
| | | AALD = 1.83 |
| 1 | 11 | |
| 2 | 8 | |
| 3 | 4 | |
| 4 | 2 | |
| 6 | 1 | |

These AALD values were tabulated for all 180 HPCT pairs and analysed for their impact upon 2 year patient survival.

### Example 3

STR determination is commonly used routinely in forensic science, paternity testing and post stem cell transplant monitoring for chimerism. The polymerase chain reaction method used in the invention to amplify STR products from donor and recipient blood or saliva samples is as described by the Eurochimera Consortium and published in Lion et. al.¹³.

It will be appreciated by a person skilled in the art that any appropriate method for STR determination from a patient sample may be used. It will be further appreciated by the skilled person that any optimisations, modifications and adaptations to the method of the present invention are within the scope of the invention.

Statistical Analysis:

Statistical analysis used Kaplan Meier estimates for survival looking at the impact of AALD values on overall stem cell transplant survival. Binary logistic regression and Cox Proportional Hazards were used to compare the influence of other transplant variables that may have acted as confounders to the impact of AALD values. Log Rank-Mantel Cox analysis for chi-square and probability were used to estimate the statistical significance of observed survival differences. Analysis was performed using IBM, SSPS software version 19.

It has been reported that higher severity of GVHD and shorter 5-year overall survival exists in patients (HLA matched sibling donor [MSD] transplants) displaying more than 10 (from 15) highly informative microsatelite (Msats) mismatches with respect to their donor (Alcoceba et. al.)⁹ These patterns have been replicated in for 52 MSD transplants but are not seen in 128 unrelated paediatric transplants where all transplant pairs had more than ten STR mismatches. The AALD values for MSD transplants overlap with those of both HR-MUD transplants and MMUD transplants for values below 1.6 in this study. The AALD values for both HR-MUD transplants and MMUD transplants show the same range and pattern of AALD values (FIG. 2.) Within the red oval in FIG. 2, the dead patients outnumber the alive patients—this is not seen at any other part of the continuum. The range of values seen in HLA matched sibling transplants, HLA HR-MUD transplants and HLA MMUD transplants are shown. The two year overall survival (OS) for all patients in this transplant cohort was 70%.

The AALD values between donor and recipient were calculated using 15 STR loci which generated an index of values that were used as a proxy for genome wide genetic difference between donor and recipient. These values formed a continuum of genetic variation for the transplant pairs examined. The higher values on this continuum associated with poor transplant outcome (FIGS. 2, 3, 4, 5 and 6).

Analysis of the data for OS in transplant pairs with AALD values above 1.9 as shown in the Kaplan-Meier overall survival estimate for (disease diverse) 180 paediatric transplant pairs in FIG. 3 it was shown that the patient overall survival is 43.7%. This compares with an OS of 72% for patients with AALD values of less than 1.9 and the difference reaches statistical significance (FIG. 3., p=0.01). From the alive to dead ratio of patients above and below a range of AALD values (FIGS. 2 and 4), it can be seen that the ratio of alive to dead progressively decreases as the AALD values increase such that the ratio inverts above AALD values of 1.8.

The difference in 2 year survival for recipients transplanted with donors yielding AALD values above and below the value of 1.8 reached statistical significance. Patients with values above 1.8 had significantly lower survival than those with values below 1.8.

A total of 180 paediatric transplants were analysed for overall survival above and below differing values of the AALD continuum. These are illustrated in FIG. 4A to D. As the AALD value increases the ratio of patients alive/dead alters and the observed difference in outcome reaches significance at a AALD value of 1.8 and above.

AALD values of below and above 1.4 showed no significant correlation with mortality 27% vs 34% (p=0.3). AALD values of below and above 1.6 also showed no significant correlation with mortality 28% vs 37% (p=0.2). However, below and above a score of 1.8 had an overall mortality of 28% vs 44% (p=0.04). If a score of 1.9 is used then the mortality below and above this value is 28% and 56% respectively (FIG. 4., p=0.01). This study was extended to include 99 adult transplant pairs, totalling 279, where AALD scores were calculated. FIG. 5, illustrates the Kaplan Meier estimate for survival in this extended study.

Where within the transplant pairs a homogeneous cohort of patients were examined, it was found that there were 77 high resolution HLA matched paediatric acute lymphoblastic leukeamic (ALL) transplant pairs. FIG. 6 compares the survival of HLA matched sibling transplants with high resolution HLA matched unrelated donors for above and below dichotomised threshold values 1.4, 1.6, 1.8 and 1.9 within this patient cohort.

AALD values offer a possible “risk index” in order to assess multiple genetic differences between donor and recipient coming from both intra and inter population ancestries. These values associate with statistically significant increased mortality above a value of 1.8 in this Western European patient cohort.

Genome wide microsatellite Average Allelic Length Discrepancy (AALD) can be used as a “risk index” where higher values directly correlate with morbidity

### REFERENCE LIST

1. Petersdorf E W. Risk assessment in haematopoietic stem cell transplantation: histocompatibility. Best. Pract. Res. Clin. Haematol. 2007; 20(2):155-70.
2. Hansen J A. Genomic and proteomic analysis of allogeneic hematopoietic cell transplant outcome. Seeking greater understanding the pathogenesis of GVHD and mortality Biol. Blood Marrow Transplant. 2009; 15(1 Suppl):e1-e7.
3. Mullighan C G, Bardy P G. New directions in the genomics of allogeneic hematopoietic stem cell transplantation, Biol. Blood Marrow Transplant. 2007; 13(2):127-44.
4. Rosenberg N A, Pritchard J K, Weber J L, Cann H M, Kidd K K, Zhivotovsky L et al. Genetic Structure of Human Populations. Science 2002; 298(5602):2381-5.
5. Slatkin M. A measure of population subdivision based on microsatellite allele frequencies, Genetics 1995; 139(1):457-62.
6. Li S, Kawata H, Katsuyama Y, Ota M, Morishima Y, Mano S et al. Association of polymorphic MHC microsatellites with GVHD, survival, and leukaemia relapse in unrelated hematopoietic stem cell transplant donor/recipient pairs matched at five HLA loci. Tissue Antigens 2004; 64(3):362-8.
7. Malkki M, Single. R, Carrington M, Thompson G, etersdorf E. MHC microsatellite diversity and linkage disequilibrium among common HLA-A, HLA-B, DRB1 haplotypes: implications for unrelated donor hematopoietic transplantation and disease association studies. Tissue Antigens 2005; 66(2):114-24.
8. Tiercy J M. Immunogenetics of hematopoietic stem cell transplantation: the contribution of microsatellite polymorphism studies. Int. J. Immunogenet. 2011; 38(5):365-72.
9. Alcoceba M, Balanzategui A, Diez-Campelo M, Martin-Jimenez P, Sarasquete M E, Chinon M C et al. Clinical and Prognostic Value of Discrepancies in Microsatellite DNA Regions Between Recipient and Donor in Human Leukocyte Antigen-Identical Allogeneic Transplantation Setting. Transplantation 2008; 86(7):893-900.
10. Ramakrishnan U, Mountain J L. Precision and accuracy of divergence time estimates from STR and SNPSTR variation. Mol. Biol. Evol. 2004; 21(10):1960-71.
11. Bradburn M J, Clark T G, Love S B, Altman D G. Survival analysis part II: multivariate data analysis—an introduction to concepts and methods. Br. J. Cancer 2003; 89(3):431-6.
12. Bradburn M J, Clark T G, Love S B, Altman D G. Survival analysis Part III: multivariate data analysis—choosing a model and assessing its adequacy and fit. Br. J. Cancer 2003; 89(4):605-11.
13. Lion T, Watzinger F, Preuner S et al. The EuroChimerism concept for a standardized approach to chimerism analysis after allogeneic stem cell transplantation. Leukemia 2012; 26:1821-1828.

1. A method for selecting a donor for a recipient in need of a transplant comprising the steps of taking a sample from the donor and recipient, extracting the DNA, measuring the short tandem repeat allele lengths in the donor and recipient DNA, determining the difference in length between the donor and recipient alleles and selecting the donor based on the difference in length.

2. A method according to claim 1 wherein the donor and recipient are HLA matched.

3. A method according to claim 1 or 2 wherein the donor and recipient alleles are paired according to length, prior to determining the difference in length.

4. A method according to claim 3 wherein the donor is selected based on the smallest difference in short tandem repeat alleles in a given pair.

5. A method according to any preceding claim wherein the donor material is selected from blood, saliva, an organ, cells, tissues, umbilical cord blood cells and other haematopoietic progenitor cells from adult or paediatric donors.

6. A method according to any preceding claim wherein the length difference is assigned a value and falls above a predetermined threshold value of 1.7 when the donor and recipient are unrelated.

7. A method according to any one of claims 1 to 5 wherein the length difference is assigned a value and falls within a predetermined threshold value range of 0.5 to 0.8 when the donor and recipient are related.

8. A method according to any preceding claim for determining the relative genetic divergence between a donor and recipient ancestry.

9. A method according to any preceding claim for determining the risk of organ failure in a recipient following transplantation.

10. A method according to any preceding claim wherein the recipient is a paediatric or an adult patient.

11. A method according to any preceding claim wherein the selection of donor and recipient is based on the value that is indicative of a higher survivability of the recipient.

12. A device comprising a means for measuring the short tandem repeat allele length sizes of the donor and recipient, ordering the alleles in donor recipient pairs according to length, determining the difference in length between the pairs, assigning a value to the difference, determining threshold value ranges for the length differences, determining whether the length difference of a pair falls within the threshold value and thereby determining the appropriate donor for a recipient.

13. A device according to claim 12 wherein the large sized donor allele is paired with the large sized recipient allele and the smallest sized donor allele is paired with the smallest sized recipient allele.

14. A device according to claim 12 or 13 wherein the donor and recipient are matched when the determined value is at or above the threshold value.
